(19) United States
(12) Patent Application Publication
(10) Pub. No.: US 2006/0241425 A1
(43) Pub. Date: Oct. 26, 2006

(54) Ultrasound detection

(75) Inventor: Elizabeth Ann Payne, Dorchester (GB)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Qinetiq Limited

(21) Appl. No.: 10/551,087

(22) PCT Filed: Mar. 29, 2004
(86) PCT No.: PCT/GB04/01331

§ 371 Date: Sep. 27, 2005

(51) Int. Cl. A61B 8/00

(52) U.S. Cl. 600/437

(57) ABSTRACT

An ultrasound detection approach utilizing a multi-resonator transducer is described. The transducer is capable of tracking a signal, for example a foetal heartbeat, without the need for an operator to displace the transducer. Depth selection and directional Doppler is also provided to maximize signal strength and to provide a more accurate measure of heart rate.

The present invention relates to the use of Doppler ultrasound for diagnostic applications, particularly although not exclusively, for medical diagnostic applications.

Doppler ultrasound has long been utilised in the investigation and classification of medical conditions including pregnancy. Typically, acoustic waves or ultrasound are generated by a transducer or transducers that have been placed in physical contact with a subject. Returns or echoes from targets within the subject are subsequently detected by the transducer and through suitable processing these echoes are used to detect motion of the subject under examination. It will be understood that the result can include a visual and/or audio representation.

Currently, known Doppler systems use an array of directive transducers in the form of ceramic discs distributed over a plane circular face and connected in parallel to provide a relatively broad beam of coverage. Since each disc is relatively large in terms of the wavelengths required, the beam diverges only slowly. This simple configuration has the attraction of giving reasonable coverage in a single output. Nevertheless, there are circumstances such as the monitoring of a foetus during pregnancy and indeed during delivery when the coverage provided by such systems compromises the ability to maintain an output of interest to the clinician, whether in the form of an audio output or a trace representative of the return from a target such as the foetal heart.

It has been suggested that in order to expand coverage it might be possible to increase the transducer face area. However, the extent to which this is possible is limited to a physical extent by the need for the transducers to maintain contact with the subject. Significantly, simply increasing the beam spread by increasing the size of the array has a detrimental effect on the signal to noise performance.

In an effort to maintain the received signal, there have been a number of suggestions for expanding the coverage of the transducer. For example, U.S. Pat. No. 6,469,422 discloses a two dimensional ultrasonic transducer array suitable for three dimensional phased array scanning. However, the practical implications of steering a beam electronically are such that there is a significant increase in both complexity and consequently cost in contrast with a fixed transducer array. As an alternative to electronically steering the beam, it might be possible to steer mechanically the transducer array. However, such an approach is not without its difficulties particularly when, unlike the electronically steered beam, the electronic return signals cannot be used as a direct input to the beam steer mechanism with the attendant mechanical compromises to performance this entails.

It will be further recognised that the difficulties posed by the need for coverage are not the only ones applicable to the efficient operation of an ultrasound transducer, particularly an ultrasound transducer intended for operation in the medical environment and more especially for monitoring the foetal heartbeat during the stages of pregnancy including childbirth.

Monitoring of the foetal heartbeat is a particular challenging environment for ultrasound and one in which, particularly during childbirth, the results of which can be critical in ensuring a positive outcome. Accordingly, the environment is one of the most significant in the field of medical ultrasound, particularly in view of the sheer number of such events which occur daily. However, it is not to say that the problems existing within this particular field do not carry over into other medical areas. Nevertheless, taking this particular environment as an example, it is to be noted that the depth of the foetal heart can vary considerably and that such variation occurs primarily due to the size of the mother. The abdominal wall may vary between about 2 and 20 cm in depth. The uterine wall is 1 or 2 cm thick. The position of the foetal heart will then be in the region of 5 to 10 cm below this. In most cases, the abdominal wall is more likely to be thin rather than thick. Thus, the foetal heart will therefore typically be about 10 cm below the surface of the mother. However, as the prevalence of obese women increases, the typical depth is likely to increase.

The usual position for the foetus is head down, with its spine to one side. A clinician or more likely a midwife carries out palpation to determine its position. Generally, a good position for the transducer provides a path through the back of the left shoulder of the foetus. This has been found to provide a clear signal. The midwife listens for a clean, relatively sharp sound rather than whooshing noises. For the most part, the midwife is to listening for the heart valves opening and closing, rather than the noise associated with any kind of blood flow.

The baby may be to either the left or the right. In addition, it may be breech or it may be spine to spine. This gives a large range of potential positions. The normal position of the transducer on the surface of the mother is therefore below the belly button, and to one side.

Usually, the foetus will move around during labour. The movement is fairly slow such that slight changes in the angle of the transducer can improve the signal received. When the second stage of labour is reached and the baby travels down the birth canal, the head first engages, and turns around so that it is delivered facing backwards for a standard delivery. The shoulders and the rest of the body twist round in turn. This movement may be short, through about 90°, or the baby may go the long way round, and turn all the way through 270°.

It will be readily apparent that the heart rate should be monitored right through birth. As the baby descends, the transducer will have to be moved downwards, typically to just above the pelvic bone, and tilted so the it looks down through the pelvis, and follows the path of the baby. This is quite awkward as there is a lot of movement involved.

During prenatal examinations and labour, prior art transducers must be repositioned in order to maintain the foetal heart signal. A method by which the transducer requires less repositioning would be advantageous.

The typical heart rate is 140 bpm (beats per minute). This can decrease to 50 bpm, and increase to 220 bpm. Outside this range, either above or below, indicates that the baby is in serious trouble. Current monitors do not indicate rates outside the specified limits (50 to 210 bpm), and may wrap the values round, giving a false reading of a safe rate.

One particular approach at least partially seeking to address the problems inherent in the above environment, is that set out in U.S. Pat. No. 4,143,650. In this document there is disclosed the application of ranging and directional Doppler techniques. By ranging is meant the gating of returns or echoes such that returns received outside of a time window corresponding to the time of flight of returns from a target or targets within a depth or section of the subject are rejected in the formation of target return. By directional Doppler is meant the ability to discriminate the returns from a target moving in a sense either towards or away from the transducer.

According to one aspect of the present invention, there is provided an ultrasound signal tracking method comprising selecting signals from a first subset of resonators chosen from a plurality of resonators forming a transducer array and subsequently selecting signals from a second subset of the plurality of resonators responsive to a comparison between returns received at one or more resonators in the first subset with returns received at one or more resonators in the second subset.

In one embodiment the ultrasound signal tracking method comprises selecting a first subset of resonators from a plurality of resonators forming a transducer array such that said first subset is centred on a resonator receiving a return meeting a predetermined condition and monitoring returns received by each of said resonators in said first subset such that when a return meeting said predetermined condition is received by another resonator in said subset other than that on which the subset is centred, a second, different subset of resonators is selected centred on said another resonator.

The predetermined condition may be a return of signal strength greater than the returns received by said other resonators in said subset.

The predetermined condition may be a correlation coefficient greater than the correlation coefficient from said other resonators in said subset, when correlated with a stored reference signal.

In some embodiments the second subset differs from said first subset; the second subset may includes at least some resonators included in said first subset; the second subset may include the first resonator.

The second subset may be centred upon said second resonator.

In one preferred embodiment at least one of said first and said second subsets comprises an hexagonal arrangement of six resonators centred around a single seventh resonator, and both of said first and said second subsets may comprise an hexagonal arrangement of six resonators centred on a single seventh resonator.

Selection of the second subset may be automated or may be effected manually.

The plurality of resonators forming the transducer array are regularly arranged, optionally in a regular hexagonal arrangement.

The method may further comprise performing phase comparison to obtain directional Doppler information.

The method may also comprise performing depth selection.

Clearly, the selection of a particular tracking algorithm is dependent on the nature of the clinical requirements of the situation in which the transducer is being utilised. In some cases, there may be a need to respond slowly to movement in the target from which the largest return is received. In other cases the ability to track rapid movements may be paramount. Advantageously, the choice of algorithm may be under operator control.

According to another aspect of the invention, there is provided an ultrasound transducer arranged to perform these methods.

In a particular embodiment there is provided an ultrasound transducer comprising a plurality of resonators and a switch operable in response to instructions from a controller to select subsets of said resonators, said controller being operable to select a first subset of resonators from said plurality of resonators such that said first subset is centred on a resonator receiving a return meeting a predetermined threshold, said controller being further operable to monitor returns received by each of said resonators in said first subset such that when a return meeting said predetermined threshold is received by another resonator in said subset other than that on which the subset is centred, said switch is instructed to select a second, different subset of resonators on said another resonator.

The resonators may be operable at a plurality of frequencies, and said controller is operable to select for a given frequency, respective first and second subsets of resonators for operation.

The resonators may be arranged on a convex surface, or in some other embodiments may be arranged at differing angles across a substantially flat surface.

In one preferred embodiment the resonators in said first and said second subsets differ.

It will be realised that resonators have inherent spatial gain and large capacitance and so are more readily interfaced to the associated electronics than would be a mechanically steered array. Advantageously, the single channel approach requires only one channel to be interfaced and processed whereas the multi-element solutions have associated multiple channels.

In covering a volume greater than that covered by prior art transducers, for example an 80° solid angle, it is desirable that the transducer footprint is of the same order of size as prior art transducers. This is not a simple comparison as prior art systems provide a coverage that diverges only slowly with range. By comparison, the present invention requires coverage which more rapidly diffuses. Whilst in a phased array, this would be achieved by suitable inter-element delays requiring additional complexity in the implementation of the transducer electronics, a simpler approach may be adopted in the present invention by placing the resonators on a suitably shaped surface.

Since steering by element selection is assumed, it is practical for individual beams to be wider than the beamwidth of single-element prior art systems. By way of example, smaller elements giving a wider beam pattern may be used.

According to yet another aspect of the invention, there is provided an ultrasound transducer system substantially as described above, wherein a signal from the transducer provides an output signal to a monitor connectable in use thereto, the output signal being derived from the resonator on which the subset of resonators is centred.

The output signal may be a directional Doppler signal.

Preferably, the system incorporates a range gating feature whereby the signal strength may be additionally maximised in respect of the depth of a target generating a return of interest. Typically, this will be a heart. Conveniently, the monitor will be a visual display unit although it may be an audio loudspeaker. In the former case, the visual display unit may display a figure indicative of heart rate. In either case, the system may include a directional Doppler processing module whereby incorrect counting of heart rate is minimised. The connection between the transducer and the monitor may be wired although advantageously a wireless connection may be provided. The selection of a particular wireless technology will depend on the data rate and electromagnetic environment in which the system operates. However, it may be that Low Power Radio Frequency (LPRF) techniques such as Bluetooth or infra-red connectivity may be particularly acceptable.

The preferred features may be combined as appropriate, as would be apparent to a skilled person, and may be combined with any of the aspects of the invention. Other advantages of the invention, beyond the examples indicated above, will also be apparent to the person skilled in the art.

In order to assist in understanding the invention, a number of embodiments thereof will now be described by way of example and with reference to the accompanying drawings, in which:

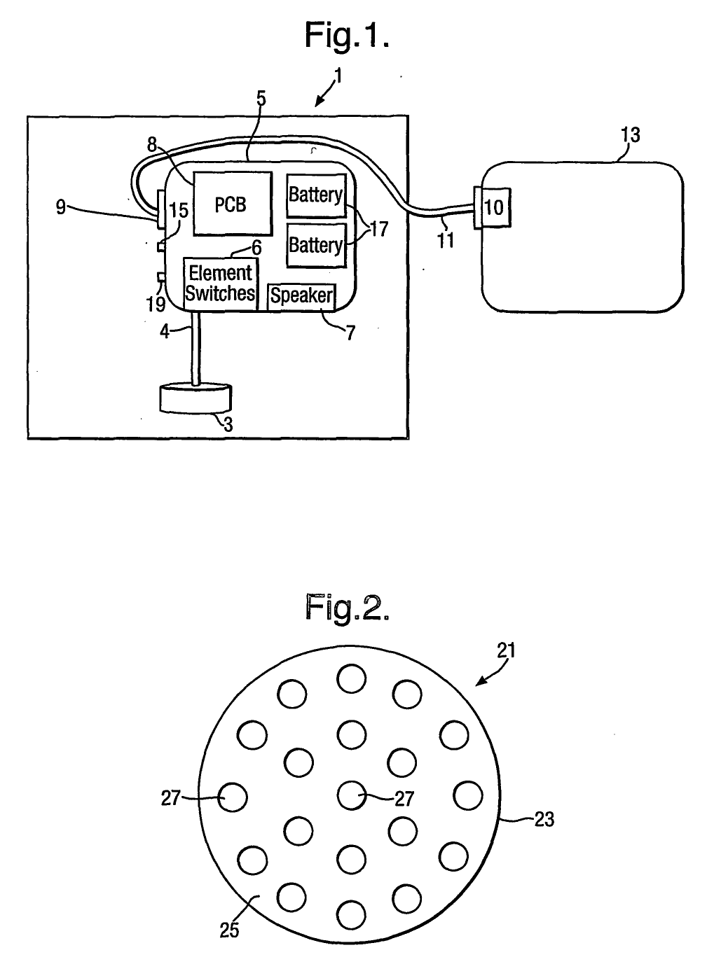

FIG. 1 is a block diagram of a system according to one aspect of the invention;

FIG. 2, is a plan view of a transducer housing according to an aspect of the invention;

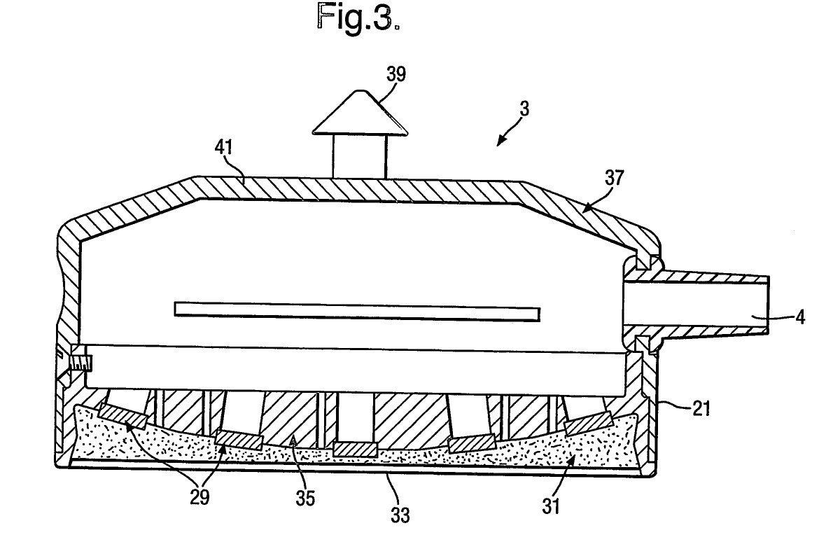

FIG. 3 is a sectional view of a transducer assembly using the housing of FIG. 2;

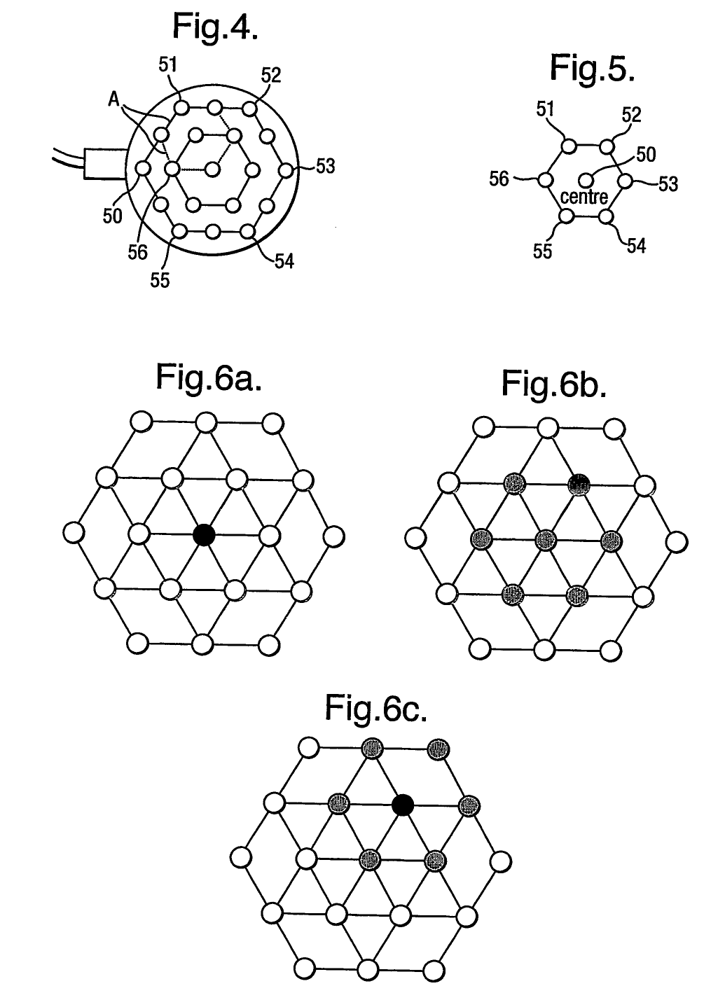

FIG. 4 is a schematic view of the transducer of FIG. 3;

FIG. 5 is a schematic view of the transducer of FIG. 3 showing the centre elements of the so-called coarse settings;

FIGS. 6a to 6c are schematic views of the transducer of FIG. 3 illustrating a search and tracking function;

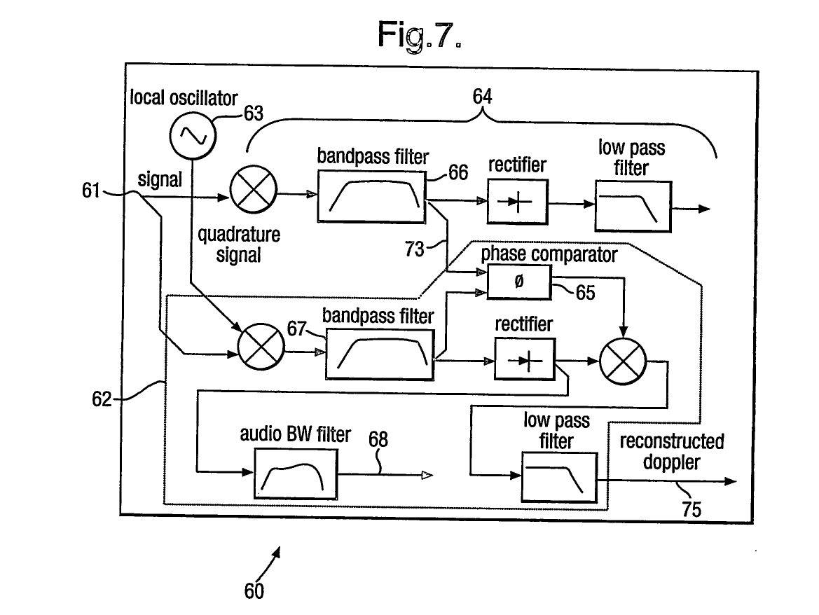

FIG. 7 is a single channel block diagram of a Doppler detection module of the transducer of FIG. 3;

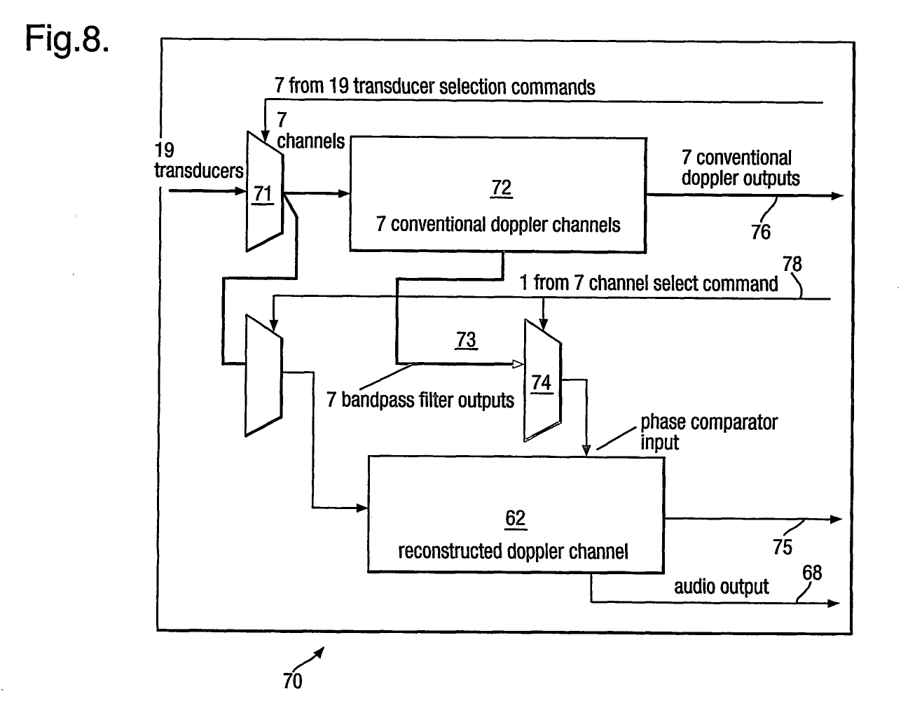

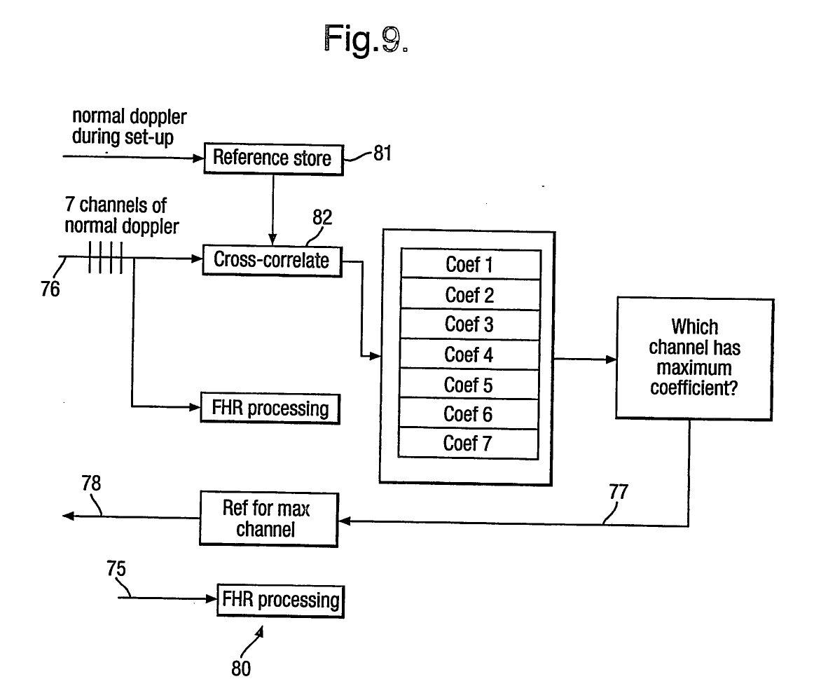

FIG. 8 is a block diagram of a direction Doppler detection architecture of the transducer of FIG. 3;

FIG. 9 is a block diagram of a tracking module of the transducer of FIG. 3;

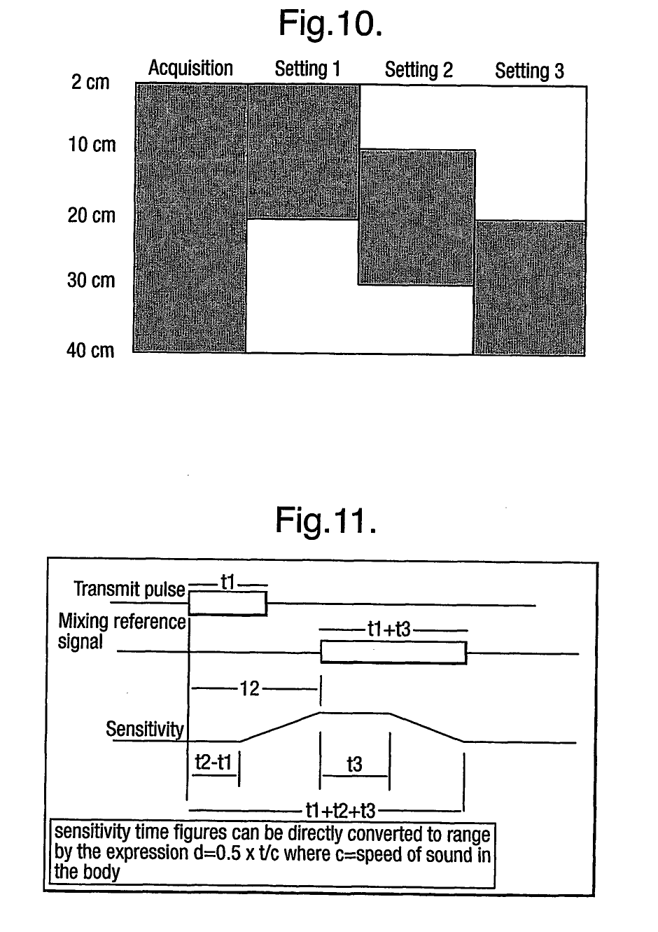

FIG. 10 is a chart illustrative of depth ranges covered by a transducer according to another aspect of the invention; and

FIG. 11 is diagram useful in understanding the correlation between time and depth sensitivity of a transducer.

With reference to the FIG. 1, there is shown a system 1 for monitoring foetal heartbeat. The system 1 comprises a transducer 3, described further below connected via a flying lead or transducer cable 4 to a control unit 5. Although shown as a separate item, the control unit may be co-located with the transducer. The control unit 5 houses a printed wiring board (PWB) 8 which provides the functionality necessary to operate the transducer 3 in conjunction with a switching unit 6 and to deliver audio signals to a speaker 7 as well as providing data output via a connector 9 and cable 11 to a computer 13 executing signal processing software. The control unit 5 is further provided with a port 15 for charging a battery 17 which supplies the power necessary to operate the control unit 5 and transducer 3. A headset socket 19 is additionally provided to allow discreet monitoring of the audio signals in place of the speaker 7.

Turning to FIGS. 2 and 3, there is shown one embodiment of a the transducer 3 in more detail. A plastics material housing 21, typically polyurethane, (see FIG. 2) has a circumferential wall 23 extending from a convex, stepped face 25 having a plurality of apertures 27 arranged as two concentric rings at 7-degree angles from an aperture 27 at the centre of the face 25. The radius of curvature is such that there is close to a 40° angular coverage from the centre of the transducer 3. Each aperture 27 is intended as a mounting site for a ceramic resonator disc 29, typically PZT4 with a radius of 6.35 mm and thickness resonance of 1 MHz. Each disc 29 has a beam width of approximately 14°.

A polyurethane material 31 is used for a front face and the housing of the device. A thin layer of rho-c material is spread across the face 33 of the transducer 3 to create a flat surface. Syntactic foam 35 is used to back-fill the voids behind the stepped face.

As has been mentioned, the discs 29 fit in apertures or recesses 27 within the stepped face 25 Electrical connections are made from each of the discs 29 via a vero board and the cable 4 to a respective switch in the switch unit 6 of the control unit 5. The ceramic discs 29 are held in place by an adhesive. The diameter of the housing 21 is around 8 cm with a total thickness of about 4 cm. A lid or closure 37 is provided with a button or stud 39 on an external surface 41 that enables the transducer 3 to be secured using a known foetal heart monitor belt (not shown).

In accordance with one embodiment of the invention, a manual switching device 6 is to be used. The nineteen signal leads from the transducer 3 are lead, as has been described, to a switching box 6. This box includes two switches. The ‘coarse’ switch moves a seven-element hexagonal search pattern around the main 19-transducer head pattern as will be more clearly understood by reference to FIGS. 4 and 5. In FIG. 4, one such selection is shown by the chain line A. Thus, the switch is so configured that in addition to selecting an hexagonal array of discs surrounding and including the central disc, the switch can separately select an hexagonal array of discs adjacent to one of the hexagonal transducer array vertices of the entire array. The coarse switch selects a 7 element hexagon, which may be centred on any of the vertices (50-56) shown in (FIG. 5). A second ‘fine’ switch enables either all, or one of, the elements within the chosen hexagon to be chosen.

In use, an operator is able to manually input a selection of the seven-element hexagonal search pattern whereby the strongest signals from the transducer may be derived through listening to the audio signal.

In another embodiment of the invention (see FIGS. 6a to 6c), the selection of the seven-disc hexagonal search pattern is carried out through a switching and tracking function provided by software and/or hardware placed on the PWB 8. In an initial acquisition phase (FIG. 6a), the switching function will default to the operation of the single central disc (shown in black on FIG. 6a). An operator will then manually acquire a heart signal to be tracked by moving the transducer over the surface of the mother. Once acquired, the switching function activates the further six discs (shown in gray on FIG. 6b) surrounding the central disc to give the seven disc hexagonal search pattern (FIG. 6b). Subsequently, by monitoring any movement or drift in the strongest signal from the seven discs (disc shown in black on FIGS. 6b and 6c), the tracking functions causes the switching function to activate a new set of seven discs (FIG. 6c) centred on the disc receiving the strongest signal. It will be recognised that the updating of the seven disc hexagonal search pattern may not occur on every cycle of transducer operation but may be delayed until a trend can be established in the tracking of the strongest return signal.

Irrespective of whether the tracking of the strongest signal is carried out by manual or automated means, as set out above, the control unit also provides directional Doppler detection. FIG. 7 shows a single channel directional Doppler detection module 60 according to one aspect of the present invention. This module 60 uses a signal 61 and a second auxiliary channel 62 to process the input signal 61 in conjunction with an output from a local oscillator 63 in quadrature to the original oscillator output used in the conventional Doppler channel 64. A phase comparator 65 is used to detect when the Doppler shift changes between positive and negative owing to the fact that the phase of the output from bandpass filters 66,67 in the respective main and auxiliary channels 64,62 rotates with respect to each other by 180° when the Doppler shift changes between positive and negative. The phase comparator 65 output indicative of such an event is then used to invert the phase of the rectified output from the bandpass filter 67 of the auxiliary channel 62 to produce a synthesised polarity conscious signal for use in subsequent signal processing and in particular extraction of the foetal heart rate (FHR).

The single channel directional Doppler detection module 60 shown in FIG. 7 is the basis of the directional Doppler architecture 70 shown in FIG. 8. Turning to FIG. 8, the signals from the seven disc hexagonal search array are selected 71 and seven conventional Doppler channels are formed 72. These may then be utilised in the automated tracking of the strongest signals in a tracking module 80 shown in FIG. 9, the operation of which is described further below. In addition one of the seven band-pass filtered outputs 73 of the conventional Doppler channels is selected 74 and provided as an input to a phase comparator 65 in a reconstructed Doppler channel 62.

In order to provide an audio feed for the operator, as there is only need for audio from the best correlated channel, the audio feed 68 is taken from the same rectified output of the auxiliary channel.

The tracking module 80 referred to above and shown in FIG. 9, includes a reference store 81 which is provided with data retrieved from central disc during the initial operator signal acquisition phase set out above. The data held in the reference store 81 is utilised by a cross-correlator 32 that receives as input the signals from the seven channels of normal Doppler, that is non-directional, received from the discs currently providing the hexagonal seven disc search array. The channel having the maximum coefficient as determined by the cross-correlator is then identified 77 and this is selected as the centre channel, for the next cycle of transducer operation. If there is a change in the centre channel, then as was set out previously, there will be a corresponding change in the discs used to form the seven disc search array.

In a further embodiment of the invention, depth control of the area covered by the transducer is provided to supplement the tracking capability of the transducer. Whilst depth control may be manual in the sense that operator is able to select ranges of particular depth in accordance with feedback from the target returns, preferably depth control is automatic. FIG. 10 shows the options available for depth selection including an acquisition phase. Table 1 below provides transmit and receive timings based on a speed of sound in tissue of 1540 m/s.

TABLE 1
Prf: 1600 Hz cycle time: 0.000625 s

| start depth | stop depth | % start | % stop | % start | % stop |
|---|---|---|---|---|---|
| (cm) | (cm) | transmit | transmit | receive | receive |
| 0.02 | 0.4 | 0 | 15 | 20 | 98 |
| 0.02 | 0.2 | 0 | 15 | 20 | 57 |
| 0.1 | 0.3 | 0 | 15 | 36 | 78 |
| 0.2 | 0.4 | 0 | 15 | 57 | 98 |

The Doppler channels are a self-heterodyning system, whereby a transmission pulse is made at a set frequency utilising the same oscillator used in the directional Doppler detection module. The return signal is mixed with a phase and frequency identical signal for defined periods of time, the Doppler difference frequencies then being extracted via bandpass frequencies. The extracted signals vary in time (measured with respect to the timing of the transmission pulses) with the depth of the target within the body.

The basic correlation between time and depth sensitivity is shown in FIG. 11. In all cases the transmit signal is repeated at a pulse repetition frequency (prf) of 1.6 kHz. It is gated on for 93 usec (15% of the cycle time), and the mixing reference signal is gated on for times corresponding to the depths indicated in FIG. 9 as shown in Table 2 below (all times as referred to the start of a transmission pulse):

TABLE 2

| Mode | Start time | End time |
|---|---|---|
| Acquisition | 125 μsec | 612 μsec |
| setting 1 | 125 μsec | 362 μsec |
| setting 2 | 225 μsec | 481 μsec |
| setting 3 | 362 μsec | 612 μsec |

The output from the mixer stage of the Doppler channel is effectively sampled at the prf. The following band-pass filter, typically with a bandwidth of 100 Hz-400 Hz, removes the 1.6 kHz sampling artifacts as well as the twice-frequency sum-and-difference products, yielding a clean doppler signal from the depth range indicated.

The selection of the different mixing reference signal times is controlled via a logic circuit which can accept direct switching control or remote instructions via a control signal from the computer carried via the cable. The different settings are also re-programmable so that different timings for the four settings can be established if found necessary or advantageous during calibration.

For normal deployment the setting will be to Acquisition mode until a strong signal is found, using the transducer selection settings and heart-rate extraction processing. Once a strong signal has been established the depth settings will be adjusted for best signal. However, it is anticipated that with experience the clinician carrying out the initial adjustments will be able to set the depth settings directly by observation of the patient.

It will be appreciated that the signal processing required to generate a display of the foetal heartbeat may carried out on a processing board and/or within software loaded on the computer. The input to the signal processing is simply the reconstructed Doppler output described above in relation to the directional Doppler system architecture. The signal processing techniques themselves are well known to those skilled in the art and generally comprise the steps of removing dc offset from the signal, rectification, low pass filtering and finally re-sampling before display on a monitor. The foetal heart rate itself is derived from auto-correlation of the signal with the correlation coefficient giving a measure of confidence in the foetal heart rate figure.

Whilst the preferred embodiments described above typically make use of subsets of resonators comprising seven resonators in a hexagonal arrangement, it will be apparent to the person skilled in the art that other subsets may be used in which the selected subsets may have different physical arrangements relative to the selected element. For example sets of three resonators arranged in a triangle, or sets of four resonators, again in a triangular arrangement but with an additional resonator in the centre, may be used. In some arrangements—for example the 3-element triangular arrangement—there may be no central element. In such arrangements then, the subset of elements may simply be chosen so that the selected element is any one of the elements of the subset.

Furthermore it is not essential that the layout of successively selected subsets be uniform. So, for example, in an arrangement in which hexagonal subsets of seven elements are selected centred on an inner element, “partial” hexagons may be selected when the best signal is received from an outer edge element. In those cases the partial hexagonal subset may comprise, for example, four elements where the central element is located at an edge apex, or five elements where the central element is located on the outer rim other than at an apex.

It will also be apparent that the actual layout of the elements within the transducer array may vary. For example a square array of elements may be used in place of the triangular/hexagonal orange and illustrated, and in some cases the distribution may not be uniform.

It will also be apparent to the person skilled in the art that the selection of successive subsets may be based on comparisons between signal returns received at one or more resonator in each subset.

In all cases the general principle remains the same however: if the source of the best signal shifts, then the currently selected subset of elements moves broadly in the direction leading from the element which formerly provided the best signal towards the element now identified as receiving the best signal.

Any range or device value given herein may be extended or altered without losing the effect sought, as will be apparent to the skilled person for an understanding of the teachings herein.

1. An ultrasound signal tracking method comprising selecting signals from a first subset of resonators chosen from a plurality of resonators forming a transducer array and subsequently selecting signals from a second subset of the plurality of resonators responsive to a comparison between returns received at one or more resonators in the first subset with returns received at one or more resonators in the second subset.

2. An ultrasound signal tracking method comprising selecting a first subset of resonators from a plurality of resonators forming a transducer array such that elements of said first subset are in a predetermined physical arrangement relative to a first resonator receiving a return meeting a predetermined condition, and monitoring returns received by each of said resonators in said first subset such that when a return meeting said predetermined condition is received by a second resonator in said subset other than said first resonator, a second subset of resonators is selected in a predetermined physical arrangement relative to said second resonator.

3. A method as claimed in claim 2, wherein said predetermined condition is a return of signal strength greater than the returns received by said other resonators in said subset.

4. A method as claimed in claim 2, wherein said predetermined condition is a correlation coefficient greater than the correlation coefficient from said other resonators in said subset, when correlated with a stored reference signal.

5. A method as claimed in 1, wherein said second subset differs from said first subset.

6. A method as claimed in 1, wherein said second subset includes at least some resonators included in said first subset.

7. A method as claimed in 1, wherein said second subset includes said first resonator.

8. A method as claimed in 1 in which said second subset is centred upon said second resonator.

9. A method as claimed in 1, wherein at least one of said first and said second subsets comprises an hexagonal arrangement of six resonators centred around a single seventh resonator.

10. A method as claimed in 1, wherein both of said first and said second subsets comprises an hexagonal arrangement of six resonators centred on a single seventh resonator.

11. A method according to claim 1 in which selection of the second subset is automated.

12. A method according to claim 1 in which selection of the second subset is effected manually.

13. A method according to claim 1 in which the plurality of resonators forming the transducer array are regularly arranged.

14. A method according to 1 in which the plurality of resonators forming the transducer array are arranged in a regular hexagonal arrangement.

15. A method according to 1 further comprising performing phase comparison to obtain directional Doppler information.

16. A method according to 1 further comprising performing depth selection.

17. An ultrasound transducer arranged to perform the method of claim 1.

18. An ultrasound transducer according to claim 17 comprising a plurality of resonators and a switch operable in response to instructions from a controller to select subsets of said resonators, said controller being operable to select a first subset of resonators from said plurality of resonators such that said first subset is centred on a resonator receiving a return meeting a predetermined condition, said controller being further operable to monitor returns received by each of said resonators in said first subset such that when a return meeting said predetermined condition is received by another resonator in said subset other than that on which the subset is centred, said switch is instructed to select a second, different subset of resonators centred on said another resonator.

19. A transducer as claimed in claim 18, in which the resonators are operable at a plurality of frequencies, and said controller is operable to select for a given frequency, respective first and second subsets of resonators for operation.

20. A transducer as claimed in claim 17, wherein the resonators are arranged on a convex surface.

21. A transducer as claimed in claim 17, wherein the resonators are arranged at differing angles across a substantially flat surface.

22. A transducer as claimed in claim 17, wherein the resonators in said first and said second subsets differ.

23. An ultrasound transducer system arranged to perform the method of claims 1.

24. An ultrasound transducer system comprising a transducer in accordance with claim 17, wherein a signal from the transducer provides an output signal to a monitor connectable in use thereto, the output signal being derived from the resonator based upon which the subset of resonators was selected.

25. A system as claimed in claim 24, wherein the output signal is a directional Doppler signal.

26. A system as claimed in claims 24, wherein the output signal is maximised through the application of range gating to the output signals of the subset of resonators.

27. A system as claimed in claim 24, wherein the transducer is wirelessly connected to the monitor.
